(19) United States
(12) Patent Application Publication
(10) Pub. No.: US 2009/0254050 A1
(43) Pub. Date: Oct. 8, 2009

(54) VALVE

(76) Inventor: Michael Reinhard Bottcher, Constantia (ZA)

Correspondence Address: STITES & HARBISON PLLC, 1199 NORTH FAIRFAX STREET, SUITE 900, ALEXANDRIA, VA, 22314, US

(21) Appl. No.: 11/722,305

(22) PCT Filed: Dec. 9, 2005
(86) PCT No.: PCT/IB05/54144

§ 371 Date: Jun. 9, 2009

(30) Foreign Application Priority Data
Dec. 21, 2004 (ZA) 2004/10257
Dec. 21, 2004 (ZA) 2004/10259
Jan. 19, 2005 (ZA) 2005/00504

(51) Int. Cl. A61M 39/22

(52) U.S. Cl. 604/256

(57) ABSTRACT

Valve (10) including housing (12) having first opening side (18) and second opening side (20); closing element (22 made of deformable material, located between first and second opening sides and adapted to prevent communicatio between first and second opening side in inoperative state and adapted to allow communication between first and second opening side in operative state. Closing element may be integrally formed with housing. Housing and/or closing element made of liquid plastics material such as liquid silicone. Valve adapted to allow object such as need or other medical device, to penetrate closing element in operative state and allow closure of closing element in inoperative state.

### FIELD OF INVENTION

The present invention relates to a valve.

### BACKGROUND TO INVENTION

Valves are defined as devices for controlling the passage of fluid though a pipe, especially a device allowing movement in one direction only. Numerous valve types and designs are known from the prior art.

Valves are also utilised in the various medical fields, in particular intravenous therapy, a method for administering fluids and/or medications directly into the venous system, usually into a patient's vein. In order to administer intravenous fluids, venous access is required and this is acquired by means of a catheter over needle.

The fluids to be administered are provided by way of tubing to the catheter. Valves for such tubing have been developed, but are either difficult to operate, i.e. require two hands or are mechanically operated and due to continuous usage lead to one or other mechanical failure.

Furthermore, needle-stick injuries is a common feature when operating known medical valves for administering fluids.

It is an object of the invention to suggest a valve which will assist in overcoming the afore-mentioned problems.

### SUMMARY OF INVENTION

According to the invention, a valve includes
(a) a housing having a first opening side and a second opening side; and
(b) a closing element, made of a deformable material, located between the first opening side and the second opening side, and being adapted to prevent communication between the first opening side and the second opening side in an inoperative state and being adapted to allow communication between the first opening side and the second opening side in an operative state.

Also according to the invention, a valve includes
(a) a housing having a first opening side and a second opening side;
(b) a closing element, made of a deformable material, located between the first opening side and the second opening side, and being adapted to prevent communication between the first opening side and the second opening side in inoperative state and being adapted to allow communication between the first opening side and the second opening side in an operative state;
(c) an output section adjacent to the second opening side and being adapted to receive an associated output docking component, such as a luer connector adapted to be connected to a medical device insert, such as a needle, a medical bottle, a syringe, a dripping chamber, an infusion flow regulator, a medical fluid connection, a medical fluid adapter, an infusion equipment, a filter, an intravenous catheter tube, a cannula, a tube and/or a dosing device; and
(d) an input section adjacent to the first opening side and adapted for receiving an insert guide having a luer connection and being adapted to be connected to a medical device insert, including a needle, a medical bottle, a syringe, a dripping chamber, an infusion flow regulator, a medical fluid connection, a medical fluid adapter, an infusion equipment, a filter, an intravenous catheter tube, a cannula, a tube and/or a dosing device.

Yet further according to the invention, a catheter includes
(a) a catheter; and
(b) a valve including a housing having a first opening side and a second opening side; and a closing element, made of a deformable material, located between the first opening side lo and the second opening side, and being adapted to prevent communication between the first opening side and the second opening side in an inoperative state and being adapted to allow communication between the first opening side and the second opening side in an operative state.

The closing element may be integrally formed with the housing.

The housing and/or the closing element may be made of liquid plastics material, such as liquid silicon.

The valve may be adapted to allow an object to penetrate the closing element in the operative state and to allow closure of the closing element in the inoperative state.

The object may be a needle or other medical device.

The closing element may include a slit and/or guiding channel.

The valve may include hardened points and/or annular collars to assist in handling the valve and/or for guiding the penetrating object through the closing element in the operative state.

The valve may include an output section adjacent the second opening side.

The output section may be adapted to receive an associated output docking component, such as a medical device insert, including a needle, a medical bottle, a syringe, a dripping chamber, an infusion flow regulator, a medical fluid connection, a medical fluid adapter, an infusion equipment, a filter, an intravenous catheter tube, a cannula, a tube and/or a dosing device.

The output docking component may include a lure lock connection, a slip lock connection and/or a thread connection.

The output docking component may be a female part of a luer connector and/or a female luer cover.

The valve may include an input section adjacent the first opening side.

The input section may be adapted to receive an associated input docking component, such as a medical device insert, including a needle, a medical bottle, a syringe, a dripping chamber, an infusion flow regulator, a medical fluid connection, a medical fluid adapter, an infusion equipment, a filter, an intravenous catheter tube, a cannula, a tube and/or a dosing device.

The input docking component may include a lure lock, a slip lock connection, a thread connection, a male part of a luer connector and/or a male luer cap.

The input section may include a piston chamber for receiving an insert guide.

The insert guide may be a male part of a luer connector and/or a male luer cap.

The insert guide may be adapted to receive various medical device inserts, such as a needle, a medical bottle, a syringe, a dripping chamber, an infusion flow regulator, a medical fluid connection, a medical fluid adapter, an infusion equipment, a filter, an intravenous catheter tube, a cannula, a tube and/or a dosing device.

The valve may include a guiding channel passing from the outside through its wall to allow a penetrating object to penetrate the closing element and enable liquid to be injected thtough the second opening side.

The guiding channel may include a polycarbonate sleeve.

The valve may be integrated into a catheter.

The valve may include a safety clip for engaging with a needle to prevent needle-stick injuries.

The safety clip may be adapted to be inserted into the valve.

The safety clip may be integrally formed with the valve.

At least one safety clip may be located in the closing element, the guiding channel of the closing element and/or in the insert guide.

The valve may be used for medical, laboratory and/or chemical purposes.

### BRIEF DESCRIPTION OF DRAWINGS

The invention will now be described by way of example with reference to the accompanying schematic drawings.

In the drawings there is shown in:

FIG. 1: a sectional side view of a valve in accordance with the invention, in the inoperative state;

FIG. 2: a sectional view of the valve shown in FIG. 1, but in operative state;

FIG. 3: a sectional side view of the housing of the valve shown in FIG. 1;

FIG. 4: a sectional side view of the housing seen along arrows IV-IV in FIG. 3;

FIG. 5: a sectional side view of the housing seen along arrows V-V in FIG. 3;

FIG. 6: a sectional side view of the housing seen along arrows VI-VI in FIG. 3;

FIG. 7: a sectional side view of the connecting member of the valve shown in FIG. 1;

FIG. 8: a sectional side view of the connecting member seen along arrows VIII-VIII in FIG. 7.

FIG. 9: a sectional side view of the insert guide of the valve shown in FIG. 1;

FIG. 10: a sectional side view of the insert guide seen along arrows X-X in FIG. 9; and

FIG. 11: an end view of the insert guide seen along arrow XI in FIG. 9.

### DETAILED DESCRIPTION OF DRAWINGS

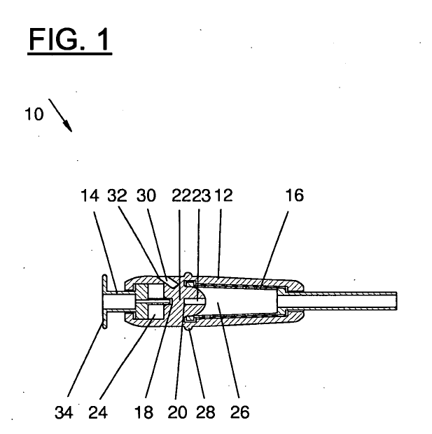

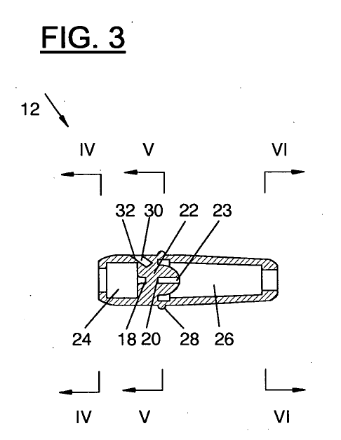

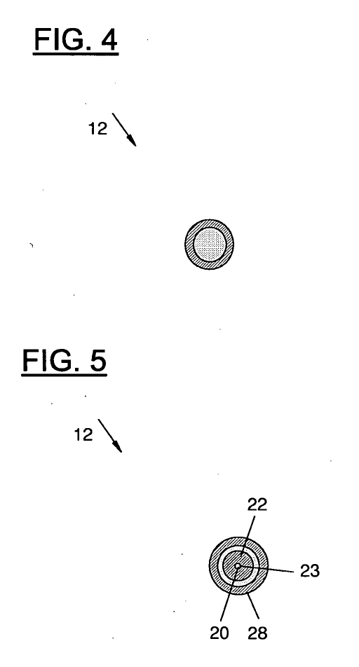

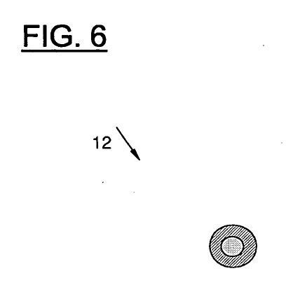

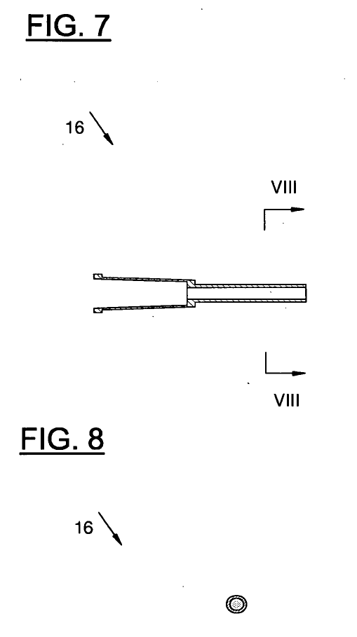

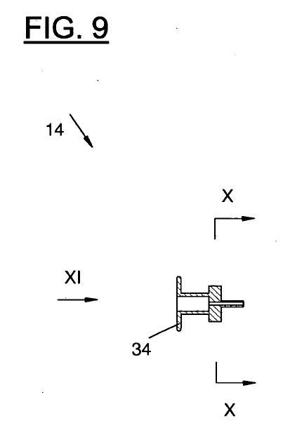

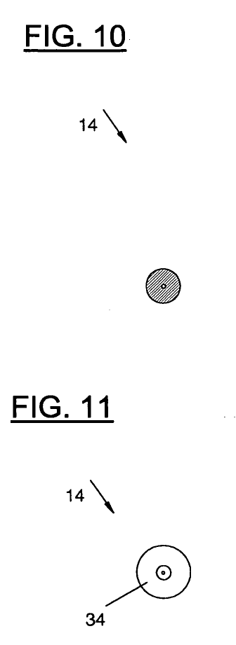

Referring to FIGS. 1 to 11 of the drawings, a valve in accordance with the invention, generally indicated by reference numeral 10, is shown. The valve 10 includes
(a) a housing 12;
(b) an insert guide 14; and
(c) a connector member or output docking member 16.

The housing 12 includes a first opening side 18 and a second opening side 20 of a closing element 22, thus being located between the first opening side 18 and the second opening side 20. A blind hole 23 is provided in the closing element 22 extending from the end face of the closing element 22 up to the second opening side 20.

The closing elementn 22 is made of a deformable material which allows it to be penetrated by an object but which returns to its original position after withdrawal of the object.

In the embodiment example, the housing 12 and the closing element 22 consist of liquid silicon plastics material.

The insert guide 14 is adapted to be located in the input section 24 adjacent to the first opening side 18 of the closing element 22. The input section 24 has the shape of a piston chamber.

The connector member 16 is designed to be located in an output section 26 located adjacent to the second opening side 20 of the closing element 22. The connector member 16 can be a cannula holder or the female part of a luer connector.

The connector member 16 in the output section 26 is adapted to receive or be connected to associated docking components, such as various medical device inserts, including needles, medical bottles, syringes, dripping chambers, infusion flow regulators, medical fluid connections, medical fluid adapters, infusion equipment, filters, intravenous catheter tubes, cannulas, tubing and/or dosing devices.

The connector member 16 in the embodiment example as shown in the Figures includes a lure lock connection, however in other embodiments it may include lure slip lock connections or thread connections.

The insert guide 14 in the input section 24 is adapted to receive or be connected to associated docking components, such as medical device inserts, needles and medical bottles.

The insert guide 14 in the embodiment example as shown in the Figures includes a lure lock connection, however in other embodiments it may include lure slip lock connections or thread connections. The insert guide 14 is adapted to receive the male part of a luer connection.

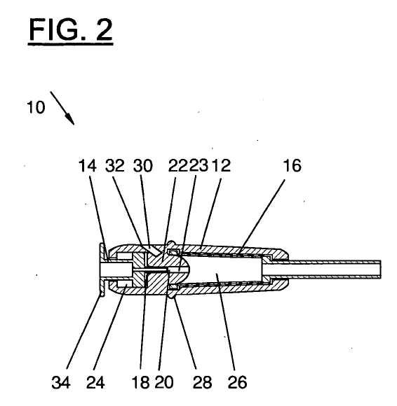

As shown in FIG. 1, the closing element 22 prevents communication between the first opening side 18 and the second opening side 20 in its inoperative state and as shown in FIG. 2 is adapted to allow communication between the first opening side 18 and the second opening side in its operative state 20.

The closing element 22 is integrally formed with the housing 12.

The insert guide 14 is made of polycarbonate material.

Thus the valve 10 in accordance with the invention allows an object (not shown) to penetrate the closing element 22 in the operative state and to allow closure of the closing element 22 in the inoperative state. The object can be a needle or other medical device.

The valve 10 includes a hardened annular collar 28 to assist in handling the valve 10 and guiding, the penetrating object through the closing element 22 in the operative state.

The valve 10 includes a guiding channel or injection port 30 passing from the outside through its wall to allow a penetrating object, such as a injecting needle, to penetrate the closing element 22 so as to enable liquid, such a pharmaceutical, to be injected into the blind hole 23.

The guiding channel or injection port 30 includes a polycarbonate sleeve 32.

The insert guide 14 is provided with a disc 34 for assisting in operating the insert guide 12, i.e. pushing, pulling, turning and locking the insert guide 14.

The valve 10 in accordance with the invention can be integrated into a catheter (not shown).

In other embodiment examples, the valve 10 is adapted to include a safety clip (not shown) for engaging with a needle to prevent needle-stick injuries. The safety clip can be located in a channel in the insert guide 14, the closing element 22 or a channel in the closing element 22.

In an example of use, a needle or medical dripping device (not shown) is inserted via the insert guide 14 into the input section 24 into the valve 10 (FIG. 1).

The insert guide 14 with the needle is pushed towards the output section 26 and the insert guide 14 together with the needle or dripping device penetrates the closing element 22 into the blind hole 23.

Once the insert guide 14 is retracted or withdrawn, the material of the closing element 22 retracts to its original position and closes off and thus seals the first opening side 18 from the second opening side 20.

The liquid silicon plastics material of the closing element 14 is not removed by the insert guide 14 nor the needle, but is merely displaced. Thus the closing element 22 automatically and independently closes after removal of the insert guide 14.

Thus no pressure or pulling forces are required to open the valve 10 in accordance with the invention and which can be manufactured easily and cost-effectively.

### LIST OF REFERENCE NUMERALS

10 Valve

12 Housing

14 Insert guide

16 Connecting member

18 First opening side

20 Second opening side

22 Closing element

23 Blind hole

24 Input section

26 Out put section

28 Annular collar

30 Guiding channel/injection port

32 Polycarbonate sleeve

34 Disc

1. A valve for medical, laboratory and/or chemical purposes, which includes
(a) a housing having a first opening side and a second opening side; and
(b) a resealable closing element, integrally formed with the housing, made of a deformable material, located between the first opening side and the second opening side, and being adapted to prevent communication between the first opening side and the second opening side in an inoperative state and being adapted to allow communication between the first opening side and the second opening side in an operative state, and being adapted to allow a penetrating object to penetrate the closing element in the operative state and to allow closure of the closing element in the inoperative state;
(c) an output section adjacent the second opening side and being adapted to be connected to at least one connection selected from the group consisting of a lure lock connection, a slip lock connection and a thread connection;
(d) an input section adjacent the first opening side, having a piston chamber, and being adapted to be connected to at least one connection selected from the group consisting of a lure lock connection, a slip lock connection and a thread connection; and
(e) an insert guide located in the piston chamber, adapted to penetrate the closing element, the insert guide being adapted to be connected to at least one connection selected from the group consisting of a male part of a luer connector and a male luer cap, the insert guide furthermore being adapted to receive at least one medical device inserts selected from the group consisting of a needle, a medical bottle, a syringe, a dripping chamber, an infusion flow regulator, a medical fluid connection, a medical fluid adapter, an infusion equipment, a filter, an intravenous catheter tube, a cannula, a tube and/or a dosing device.

2. A valve as claimed in claim 1, in which the housing and the closing element are made of liquid plastics material.

3. A valve as claimed in claim 1, in which the penetrating object is a needle or other medical device.

4. A valve as claimed in claim 1, which includes a guiding channel for guiding the insert guide.

5. A valve as claimed in claim 1, which includes at least one component selected from the group consisting of a hardened annular collar and points to assist in fulfilling at least one function selected from the group consisting of handling the valve and for guiding the penetrating object through the closing element in the operative state.

6. A valve as claimed in claim 1, in which the output section is adapted to receive at least one associated output docking component selected from the group consisting of, a medical device insert, including a needle, a medical bottle, a syringe, a dripping chamber, an infusion flow regulator, a medical fluid connection, a medical fluid adapter, an infusion equipment, a filter, an intravenous catheter tube, a cannula, a tube and a dosing device.

7. A valve as claimed in claim 6, in which the output docking component includes at least one connection selected from the group consisting of a lure lock connection, a slip lock connection and a thread connection.

8. A valve as claimed in claim 6, in which the output docking component is at east one component selected from the group consisting of a female part of a luer connector and a female luer cover.

9. A valve as claimed in claim 1, in which the input section is adapted to receive an associated input docking component selected from the group consisting of, a medical device insert, needle, a medical bottle, a syringe, a dripping chamber, an infusion flow regulator, a medical fluid connection, a medical fluid adapter, an infusion equipment, a filter, an intravenous catheter tube, a cannula, a tube and a dosing device.

10. A valve as claimed in claim 9, in which the input docking component includes at least one component selected from the group consisting of a lure lock, a slip lock connection, a thread connection, a male part of a luer connector and a male luer cap.

11. A valve as claimed in claim 1, which includes a guiding channel passing from the outside through its wall to allow a penetrating object to penetrate the closing element and enable liquid to be injected though the second opening side.

12. A valve as claimed in claim 11, in which the guiding channel includes a polycarbonate sleeve.

13. A valve as claimed in claim 1, which is integrated into a catheter.

14. A valve as claimed in claim 1, which includes a safety clip for engaging with a needle to prevent needle-stick injuries.

15. A valve as claimed in claim 14, in which the safety clip is adapted to be inserted into the valve.

16. A valve as claimed in claim 14, in which the safety clip is integrally formed with the valve.

17. A valve as claimed in claim 14, in which at least one safety clip is located in at least one component selected from the group consisting of the closing element, the guiding channel of the closing element and in the insert guide.

18. A catheter, which includes
(a) a catheter; and
(b) a valve as claimed in claim 1.

19.-20. (canceled)
